(19) United States
(12) Patent Application Publication
(10) Pub. No.: US 2025/0359564 A1
(43) Pub. Date: Nov. 27, 2025

(54) PULSED ELECTRIC FIELD PROCESSING APPARATUS

(71) Applicant: Mitsubishi Electric Corporation, Tokyo (JP)

(72) Inventors: Takahiro INOUE, Tokyo (JP); Shingo TSUDA, Tokyo (JP); Kiriu SATO, Tokyo (JP)

(73) Assignee: Mitsubishi Electric Corporation, Tokyo (JP)

(21) Appl. No.: 18/874,101

(22) PCT Filed: Jun. 15, 2022
(86) PCT No.: PCT/JP2022/023956

§ 371 Date: Dec. 12, 2024

(51) Int. Cl. A23B 2/05; A23B 2/00; A23B 11/16; A23B 70/50

(52) U.S. Cl. CPC A23B 2/05; A23B 2/001; A23B 2/003; A23B 11/16; A23B 70/50

(57) ABSTRACT

A pulsed electric field processing apparatus includes: a pulsed power supply that generates a pulse voltage; a pair of electrodes that generates a pulse electric field; a processing chamber that is disposed between the electrodes and includes a space through which a processing target object in a liquid state passes and in which the pulse electric field is generated; a temperature computing unit that computes a processing temperature that is a temperature of the processing target object in the processing chamber on the basis of a resistance value and a preset calibration value, the resistance value being acquired from the pulse voltage and a pulse current flowing through the processing target object in the processing chamber when the pulse voltage is applied to the electrodes; and a power supply control unit that controls the pulsed power supply on the basis of the processing temperature and a preset target temperature.

### FIELD

The present disclosure relates to a pulsed electric field processing apparatus that generates a pulse electric field.

### BACKGROUND

As a technique capable of processing a processing target object such as food or beverage at a temperature lower than that of heat processing, a pulsed electric field processing technique can be mentioned.

Patent Literature 1 discloses a liquid material sterilization apparatus in which a value of a required pulse voltage is calculated, for a pulse voltage applied to a sterilization processing unit, on the basis of a diameter and a shape coefficient of a bacterial cell to be sterilized, an inter-electrode distance in the sterilization processing unit, and a destruction threshold value. The destruction threshold value is a value required to set a bacterial cell-applied voltage to a destruction reference value. The bacterial cell-applied voltage refers to a voltage applied to the bacterial cell in the sterilization processing unit in the form of a pulse voltage. The destruction reference value refers to a value of the bacterial cell-applied voltage and is a value necessary for the destruction of the bacterial cell. An output of the pulse voltage from a pulsed power supply is adjusted on the basis of the calculation result and a measurement result of the pulse voltage.

### Citation List

### Patent Literature

Patent Literature 1: Japanese Patent Application Laid-open No. 2021-114943

### SUMMARY OF INVENTION

### Problem to be Solved by the Invention

In Patent Literature 1, the pulsed power supply is controlled using a resistivity of a liquid material before flowing into the sterilization processing unit. The resistivity is obtained by a resistivity measurement device disposed upstream of the sterilization processing unit. This causes a difference between the resistivity measured by the resistivity measurement device and the resistivity of the sterilization processing unit. As a result, there is a problem in that the temperature of the liquid material cannot be accurately controlled and the sterilization quality of the liquid material is deteriorated.

The present disclosure has been made in view of the above, and an object of the present disclosure is to provide a pulsed electric field processing apparatus capable of controlling a temperature of a processing target object with high accuracy and improving the processing quality of the processing target object.

### Means to Solve the Problem

To solve the above problem and achieve an object, a pulsed electric field processing apparatus according to the present disclosure, includes: a pulsed power supply to generate a pulse voltage; a pair of first electrodes to generate a pulse electric field by being applied with the pulse voltage; a first processing chamber disposed between the pair of the first electrodes, to include a space through which a processing target object in a liquid state passes and in which the pulse electric field is generated; a temperature computing unit to compute a first processing temperature that is a temperature of the processing target object in the first processing chamber on a basis of a resistance value and a preset calibration value, the resistance value being acquired from the pulse voltage and a pulse current flowing through the processing target object in the first processing chamber when the pulse voltage is applied to the first electrodes; and a power supply control unit to control the pulsed power supply on a basis of the first processing temperature and a preset target temperature.

### Effects of the Invention

A pulsed electric field processing apparatus according to the present disclosure has an effect of controlling a temperature of a processing target object with high accuracy and improving the processing quality of the processing target object.

### BRIEF DESCRIPTION OF DRAWINGS

FIG. 1 is a schematic diagram illustrating an exemplary configuration of a pulsed electric field processing apparatus according to a first embodiment.

FIG. 2 is a cross-sectional view illustrating an exemplary configuration of a processing unit of the pulsed electric field processing apparatus according to the first embodiment.

FIG. 3 is a cross-sectional view illustrating another exemplary configuration of the processing unit of the pulsed electric field processing apparatus according to the first embodiment.

FIG. 4 is a circuit diagram illustrating an exemplary configuration of a pulsed power supply of the pulsed electric field processing apparatus according to the first embodiment.

FIG. 5 is a time chart illustrating examples of a waveform of a pulse voltage and a waveform of a pulse current of the pulsed power supply of the pulsed electric field processing apparatus according to the first embodiment.

FIG. 6 is a block diagram illustrating an exemplary configuration of a control system of the pulsed electric field processing apparatus according to the first embodiment.

FIG. 7 is a time chart illustrating a temporal change of a processing temperature computed by the pulsed electric field processing apparatus according to the first embodiment.

FIG. 8 is a schematic diagram illustrating another exemplary configuration of the pulsed electric field processing apparatus according to the first embodiment.

FIG. 9 is a schematic diagram illustrating an exemplary configuration of a pulsed electric field processing apparatus according to a second embodiment.

FIG. 10 is a schematic diagram illustrating an example of a temperature distribution of the pulsed electric field processing apparatus according to the second embodiment.

FIG. 11 is a block diagram illustrating an example of a hardware configuration that implements the control system of the first and second embodiments.

### DESCRIPTION OF EMBODIMENTS

Hereinafter, a pulsed electric field processing apparatus according to embodiments will be described in detail with reference to the drawings.

### First Embodiment

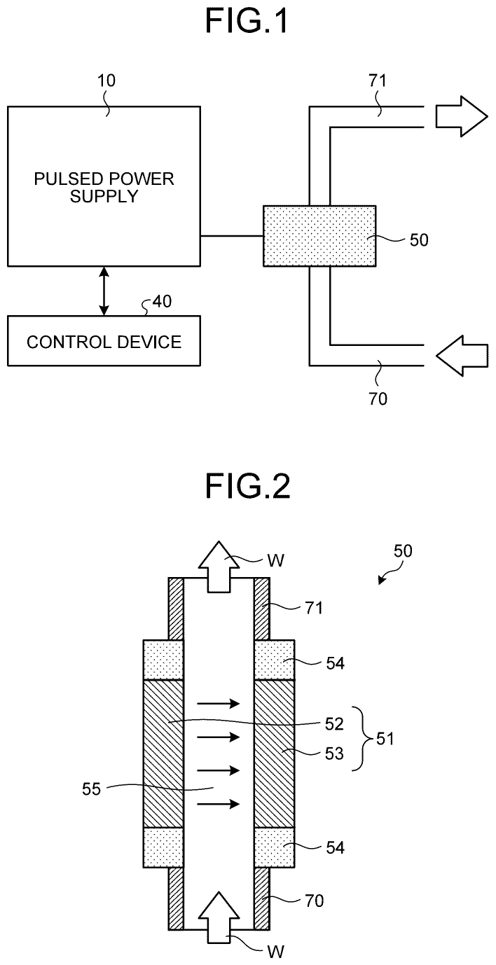

FIG. 1 is a schematic diagram illustrating an exemplary configuration of a pulsed electric field processing apparatus according to a first embodiment. The pulsed electric field processing apparatus includes a pulsed power supply 10, a control device 40, a processing unit 50, an upstream pipe 70, and a downstream pipe 71. The pulsed electric field processing apparatus executes processing including sterilization processing on a processing target object. The pulsed power supply 10 outputs a pulse voltage having a voltage of 1 kV or more and a pulse width of 100 microseconds or less, The upstream pipe 70 and the processing unit 50 are connected to each other, and the processing unit 50 and the downstream pipe 71 are connected to each other. A processing target object in a liquid state flows inside the upstream pipe 70, the processing unit 50, and the downstream pipe 71 in this order. The processing target object is, for example, a fluid such as juice or milk. The processing unit 50 is electrically connected to the pulsed power supply 10, and has a function of repeatedly applying a pulse electric field to the processing target object flowing inside the processing unit 50 using the pulse voltage output from the pulsed power supply 10. The control device 40 controls the pulsed power supply 10,

FIG. 2 is a cross-sectional view illustrating an exemplary configuration of the processing unit 50 of the pulsed electric field processing apparatus according to the first embodiment. The processing unit 50 includes an electrode 51 as a first electrode, insulating members 54, and a processing chamber 55. The electrode 51 includes a high-voltage electrode 52 and a low-voltage electrode 53, which form a pair. A pulse voltage is applied to the high-voltage electrode 52, and the low-voltage electrode 53 is maintained at ground potential. By using titanium, platinum, stainless steel, or the like as the material for the electrode 51, wear caused by the pulse voltage is mitigated. The high-voltage electrode 52 and the low-voltage electrode 53 each have a flat plate shape, and are disposed to face each other so as to sandwich the processing target object therebetween. That is, the high-voltage electrode 52 and the low-voltage electrode 53 are disposed so as to generate an electric field in a direction substantially perpendicular to a flow direction W of the processing target object.

The processing chamber 55 is a space in the processing unit 50 where an electric field is generated by the electrode 51 and the processing target object passes through. Thus, when the high-voltage electrode 52 and the low-voltage electrode 53 each have a flat plate shape and are disposed to face each other, the processing chamber 55 has a rectangular parallelepiped shape. In order to be electrically insulated from the upstream pipe 70 and the downstream pipe 71 each of which has a pipe shape and is made of metal, the high-voltage electrode 52 is connected to the upstream pipe 70 and the downstream pipe 71 with the insulating members 54 each of which is interposed between the high-voltage electrode 52 and corresponding one of the upstream pipe 70 and the downstream pipe 71. The low-voltage electrode 53 may be connected to the upstream pipe 70 and the downstream pipe 71 with the insulating members 54 each of which is interposed between the low-voltage electrode 53 and corresponding one of the upstream pipe 70 and the downstream pipe 71, or may be provided integrally with the upstream pipe 70 and the downstream pipe 71 without interposing the insulating members 54. In the former case, there is an effect of reducing electrical noise generated by the pulse voltage, and in the latter case, there is an advantage that the processing unit 50 can be downsized. By using a resin material containing fluorine or ceramic as a raw material for the insulating member 54, high heat resistance and high voltage resistance are achieved.

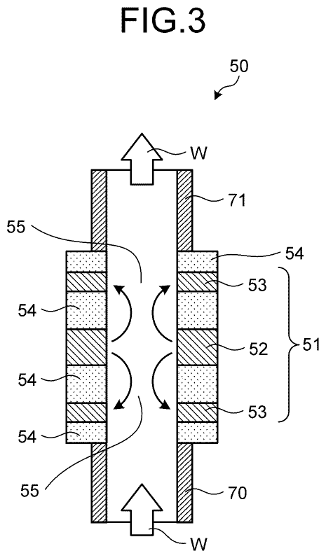

FIG. 3 is a cross-sectional view illustrating another exemplary configuration of the processing unit 50 of the pulsed electric field processing apparatus according to the first embodiment. In FIG. 3, the electrode 51 as the first electrode including the high-voltage electrode 52 and low-voltage electrodes 53 has a ring-shaped structure with the flow direction W of the processing target object as an axis. The upstream pipe 70, the insulating member 54, the low-voltage electrode 53, the insulating member 54, the high-voltage electrode 52, the insulating member 54, the low-voltage electrode 53, the insulating member 54, and the downstream pipe 71 are disposed in this order from the upstream side of the processing target object. An electric field is generated in a direction substantially along the flow direction of the processing target object. Therefore, when the high-voltage electrode 52, the low-voltage electrodes 53, and the insulating members 54 each have a ring-shaped structure and inner diameters of the high-voltage electrode 52, the low-voltage electrodes 53, and the insulating members 54 are substantially equal to each other, the processing chamber 55 has a columnar shape or a ring shape. Furthermore, since the low-voltage electrodes 53 are disposed in two places in the flow direction W of the processing target object so as to sandwich the high-voltage electrode 52, a current path flowing from the high-voltage electrode 52 toward the low-voltage electrodes 53 is divided into two. The insulating member 54 between the low-voltage electrode 53 and the upstream pipe 70 and the insulating member 54 between the low-voltage electrode 53 and the downstream pipe 71 may not be necessarily provided. When these insulating members 54 are provided, there is an effect of reducing electrical noise, and when these insulating members 54 are not provided, there is an advantage that the processing unit 50 can be downsized.

As illustrated in FIG. 2, when the high-voltage electrode 52 and the low-voltage electrode 53 are disposed to face each other, the electric field generated in the processing chamber 55 can be made spatially uniform, and an effect of reducing the unevenness of the processing can be obtained. As illustrated in FIG. 3, when the high-voltage electrode 52 and the low-voltage electrodes 53 are disposed along the flow direction W of the processing target object, the processing chamber 55 can have a columnar shape, and the processing target object can smoothly flow from the upstream pipe 70 to the downstream pipe 71 with a low pressure loss.

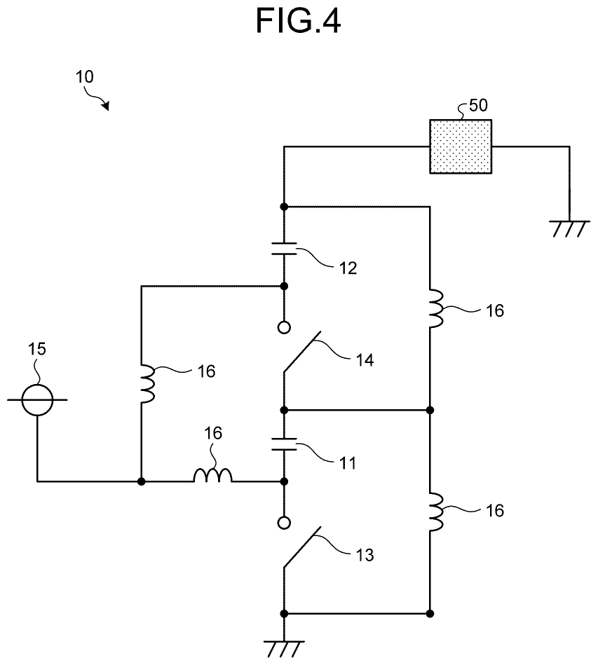

FIG. 4 is a circuit diagram illustrating an exemplary configuration of the pulsed power supply 10 of the pulsed electric field processing apparatus according to the first embodiment. In the pulsed power supply 10, a switch 13, a capacitor 11, a switch 14, and a capacitor 12 are connected in series in this order from the ground side. The high-voltage electrode 52 of the processing unit 50 is connected to the capacitor 12 by a cable or the like. In the capacitor 11, a terminal on a side of the switch 13 is a charging-side terminal, and a terminal on a side of the switch 14 is a ground-side terminal. Furthermore, in the capacitor 12, a terminal on a side of the switch 14 is a charging-side terminal, and a terminal on a side of the high-voltage electrode 52 is a ground-side terminal.

A direct-current power supply 15 generates a direct-current voltage that charges the capacitor 11 and the capacitor 12. The direct-current power supply 15 is connected to each of the charging-side terminal of the capacitor 11 and the charging-side terminal of the capacitor 12 via at least one current limiter 16. Furthermore, at least one current limiter 16 is also configured to be provided between the charging-side terminal of the capacitor 11 and the charging-side terminal of the capacitor 12. Similarly, the ground-side terminal of the capacitor 11 and the ground-side terminal of the capacitor 12 are grounded via at least one current limiter 16, and at least one current limiter 16 is provided between the ground-side terminal of the capacitor 11 and the ground-side terminal of the capacitor 12.

The pulsed power supply 10 outputs a pulse voltage in two steps, including a charging step and a discharging step. In the charging step, the capacitor 11 and the capacitor 12 are charged by the direct-current power supply 15. In the discharging step, by turning on the switch 13 and the switch 14 almost simultaneously, a charging voltage of the capacitor 11 and a charging voltage of the capacitor 12 are superimposed and output. After the charging voltage is output, the switch 13 and the switch 14 are turned off to end the discharging step. That is, a period in which the switch 13 and the switch 14 are turned on is a period of the discharging step, and corresponds to a pulse width Tp of the pulse voltage output from the pulsed power supply 10. Switching control of the switch 13 and the switch 14 is executed by the control device 40.

Semiconductor switches such as metal oxide semiconductor field effect transistors (MOSFETs) or insulated gate bipolar transistors (IGBTs) are used as the switch 13 and the switch 14. Furthermore, resistors or reactors are used as the current limiters 16. When resistors are used, there is an advantage that the capacitor 11 and the capacitor 12 can be charged with a stable voltage, and when reactors are used, power consumption can be reduced.

In the exemplary configuration of the pulsed power supply 10 illustrated in FIG. 4, two capacitors and two switches are used, but three or more capacitors and three or more switches may be used with a similar configuration. There is an advantage that a higher pulse voltage can be obtained as the number of capacitors and switches increases.

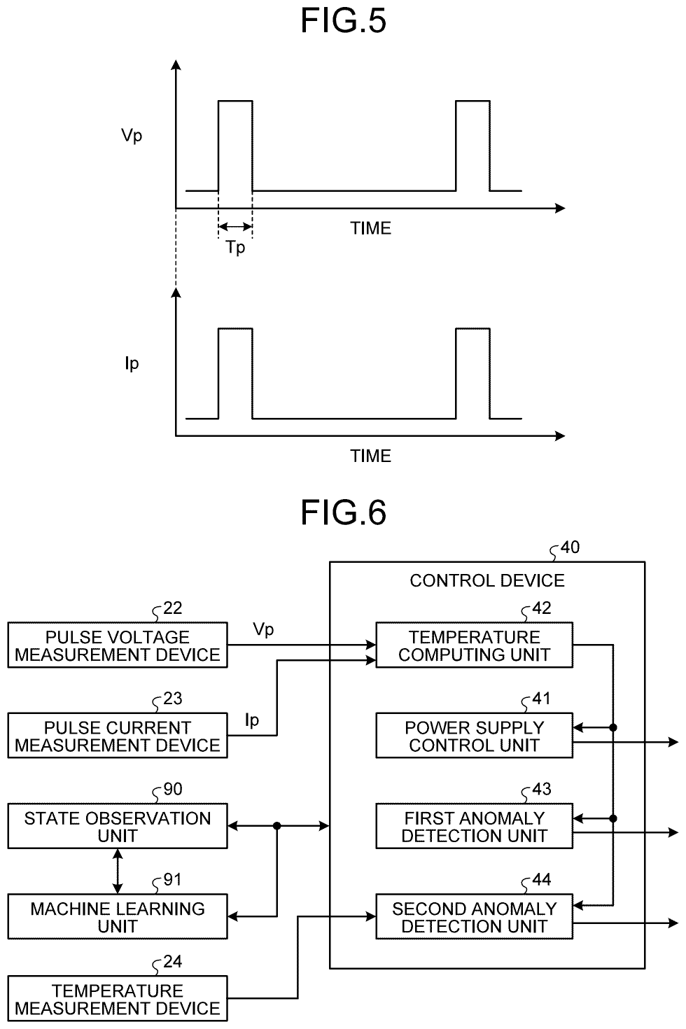

FIG. 5 is a time chart illustrating examples of a waveform of a pulse voltage Vp and a waveform of a pulse current Ip of the pulsed power supply 10 of the pulsed electric field processing apparatus according to the first embodiment. In an upper diagram of FIG. 5, the pulse voltage Vp is a voltage that the pulsed power supply 10 outputs to the high-voltage electrode 52 during the discharging step. The pulse voltage Vp has the pulse width Tp. In a lower diagram of FIG. 5, the pulse current Ip is a current flowing through the processing chamber 55 according to the pulse voltage Vp.

FIG. 6 is a block diagram illustrating an exemplary configuration of a control system of the pulsed electric field processing apparatus according to the first embodiment. The pulsed power supply 10 includes a pulse voltage measurement device 22 that measures the pulse voltage Vp and a pulse current measurement device 23 that measures the pulse current Ip. The pulse current measurement device 23 measures the pulse current flowing through the processing target object in the processing chamber 55 when the pulse voltage Vp is applied to the electrode 51. The pulse voltage measurement device 22 and the pulse current measurement device 23 repeatedly measure the pulse voltage Vp and the pulse current Ip, respectively, at intervals shorter than the pulse width Tp, and calculate the pulse voltage Vp and the pulse current Ip, respectively, using a plurality of measurement values obtained. Alternatively, by providing a capacitor for measurement, the pulse voltage Vp and the pulse current Ip may be estimated using an integrated value for the pulse width Tp. In the former case, there is an advantage that the measurement can be performed with high accuracy, and in the latter case, the apparatus can be downsized.

In order to further simplify the apparatus, the pulse voltage Vp and the pulse current Ip may be estimated from a voltage or a current inside the pulsed power supply 10. The pulse voltage Vp can be calculated using a voltage charged in the capacitor 11 or the capacitor 12. Furthermore, the pulse current Ip can be calculated from the pulse voltage Vp, the pulse width Tp, and a current that flows through the capacitor 11 or the capacitor 12 after the discharging step. The pulsed power supply 10 has a function of optionally setting the pulse width Tp and the voltage output from the direct-current power supply 15. Therefore, the pulse voltage Vp and the pulse current Ip can be estimated by measuring only the current flowing through the capacitor 11 or the capacitor 12.

As illustrated in FIG. 6, the control device 40 includes a power supply control unit 41, a temperature computing unit 42, a first anomaly detection unit 43, and a second anomaly detection unit 44. The temperature computing unit 42 computes a resistance value Rw (not illustrated) of the processing target object existing in the processing chamber 55 using the pulse voltage Vp measured by the pulse voltage measurement device 22 and the pulse current Ip measured by the pulse current measurement device 23. Furthermore, the temperature computing unit 42 computes a processing temperature tw as a first processing temperature, which is a temperature of the processing target object in the processing chamber 55, using a preset calibration value and the computed resistance value Rw. The calibration value refers to a value based on the temperature dependence of the conductivity of the processing target object, and a value corresponding to the type of the processing target object is used for the calibration value. Moreover, the temperature computing unit 42 has a function of computing the processing temperature tw in a first cycle Tc and storing the computed processing temperature tw. The processing temperature tw may be computed every time the pulse voltage Vp is output, or may be computed every time the pulse voltage Vp is output twice or more. In the former case, an effect of enhancing responsiveness can be obtained due to the short computation cycle. In the latter case, an effect of reducing the computation load and downsizing the apparatus can be obtained.

The power supply control unit 41 controls on and off of the switch 13 and the switch 14 on the basis of the processing temperature tw computed by the temperature computing unit 42 and a preset target temperature, to control the output from the pulsed power supply 10. Specifically, at least one of an upper limit value and a lower limit value is preset as a target temperature for the processing temperature tw, and control of lowering the output from the pulsed power supply 10 is performed when the processing temperature tw exceeds the upper limit value, and control of increasing the output from the pulsed power supply 10 is performed when the processing temperature tw falls below the lower limit value. When the processing temperature tw is too high, there arises a problem that the flavor, nutrients, and the like of the processing target object are impaired, whereas when the processing temperature tw is too low, the sterilization effect etc. are reduced. Therefore, by performing the above control, the processing target object can be maintained at an appropriate temperature, and the quality of the processing target object can be improved.

The control of the output from the pulsed power supply 10 is adjusted by changing at least one of the pulse voltage Vp, the pulse width Tp, and pulse frequency fp (not illustrated). If the pulse voltage Vp is changed, it is necessary to control the direct-current power supply 15, and the responsiveness is low. Therefore, high responsiveness can be acquired by controlling on and off of the switch 13 and the switch 14 so as not to change the pulse voltage Vp and to change at least one of the pulse width Tp and the pulse frequency fp.

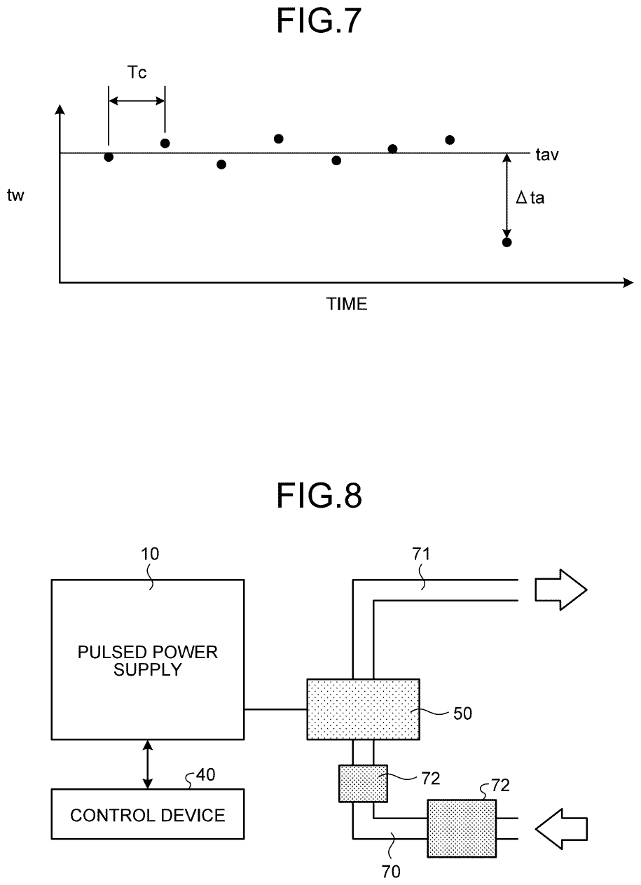

The first anomaly detection unit 43 outputs a first anomaly signal when the processing temperature tw computed in the first cycle Tc exceeds a normal temperature range that is defined by a preset set value or when a difference exceeding a set value Δtmx occurs between the processing temperature tw and an average value of the past processing temperatures tw that is stored in the temperature computing unit 42. FIG. 7 is a time chart illustrating a temporal change of the processing temperature tw computed by the pulsed electric field processing apparatus according to the first embodiment. Black dots indicate the processing temperatures tw computed every first cycle Tc. An average value tav is an average value of a plurality of past processing temperatures tw, When a difference Ata between the processing temperature tw computed present time and the average value tav exceeds the set value Δtmx, the first anomaly detection unit 43 outputs a first anomaly signal.

The temperature computing unit 42 can compute a temperature rise Δt of the processing target object in a period in which the processing target object passes through the processing unit 50. In other words, the temperature computing unit 42 can compute the temperature rise Δt that is a temperature difference between the temperature of the processing target object before passing through the processing unit 50 and the temperature of the processing target object after passing through the processing unit 50. That is, the temperature computing unit 42 can compute the temperature rise Δt of the processing target object in a period in which the processing target object passes through the processing unit 50 on the basis of a relationship between a flow rate and a physical property value of the processing target object and the power output from the pulsed power supply 10. The processing temperature tw computed by the temperature computing unit 42 represents the average temperature of the spatial temperature distribution in the processing chamber 55. Therefore, by adding about ½ of the temperature rise Δt to the processing temperature tw, a temperature twd of the processing target object downstream of the processing unit 50 can be estimated. The temperature computing unit 42 transmits the estimated temperature twd of the processing target object downstream of the processing unit 50 to the second anomaly detection unit 44.

Furthermore, the temperature measurement device 24 that measures a temperature of the processing target object is provided downstream of the processing unit 50, The temperature measurement device 24 is, for example, a thermocouple.

The second anomaly detection unit 44 periodically compares the temperature twd of the processing target object downstream of the processing unit 50 estimated by the temperature computing unit 42 with the temperature of the processing target object downstream of the processing unit 50 measured by the temperature measurement device 24. The second anomaly detection unit 44 outputs a second anomaly signal if a difference exceeding a set value occurs between the temperatures compared. By providing the second anomaly detection unit 44, whether or not the processing temperature tw computed by the temperature computing unit 42 is correct can be constantly monitored, and there is an advantage that anomaly can be detected when anomaly occurs.

Note that the temperature measurement device 24 may be provided upstream of the processing unit 50, and the second anomaly signal may be output on the basis of a comparison between the value measured by the temperature measurement device 24 provided upstream of the processing unit 50 and the temperature of the processing target object upstream of the processing unit 50 estimated by the temperature computing unit 42. Moreover, the second anomaly signal may be output on the basis of a comparison between values measured by temperature measurement devices 24 provided downstream and upstream of the processing unit 50 and temperatures of the processing target object downstream and upstream of the processing unit 50 estimated by the temperature computing unit 42.

The pulsed electric field processing apparatus of the first embodiment includes a state observation unit 90 and a machine learning unit 91. The state observation unit 90 has a function of observing a charging voltage or a charging current in the capacitor 11 or the capacitor 12 as a state variable. The machine learning unit 91 learns a normal value of the charging voltage or the charging current using a training data set created on the basis of the state variable and the physical property value of the processing target object. By creating the training data set, wear of the electrode 51 caused by aging can be considered with respect to the normal value of the charging voltage or the charging current. Furthermore, the machine learning unit 91 has a function of transmitting an anomaly notification to the control device 40 when the value of the charging voltage or the charging current deviates the normal value of the charging voltage or the charging current.

FIG. 8 is a schematic diagram illustrating another exemplary configuration of the pulsed electric field processing apparatus according to the first embodiment. In FIG. 8, heating units 72 having a function of increasing the temperature of the processing target object is provided upstream of the processing unit 50. A higher processing effect can be obtained by applying the pulse voltage Vp to the processing target object in a state where the processing target object has a higher temperature. The pulsed power supply 10 can be downsized by using both the pulsed power supply 10 and the heating unit 72, thus expectedly resulting in downsizing and cost reduction of the pulsed electric field processing apparatus.

As described above, according to the first embodiment, the temperature computing unit 42 computes the processing temperature tw that is the temperature of the processing target object in the processing chamber 55 on the basis of the resistance value Rw and the preset calibration value. The resistance value Rw is acquired from the pulse voltage Vp and the pulse current Ip flowing through the processing target object in the processing chamber 55 when the pulse voltage Vp is applied. In addition, the power supply control unit 41 controls the pulsed power supply 10 on the basis of the processing temperature tw and the preset target temperature. Therefore, the temperature of the processing target object can be controlled with high accuracy, and the processing quality of the processing target object can be improved.

### Second Embodiment

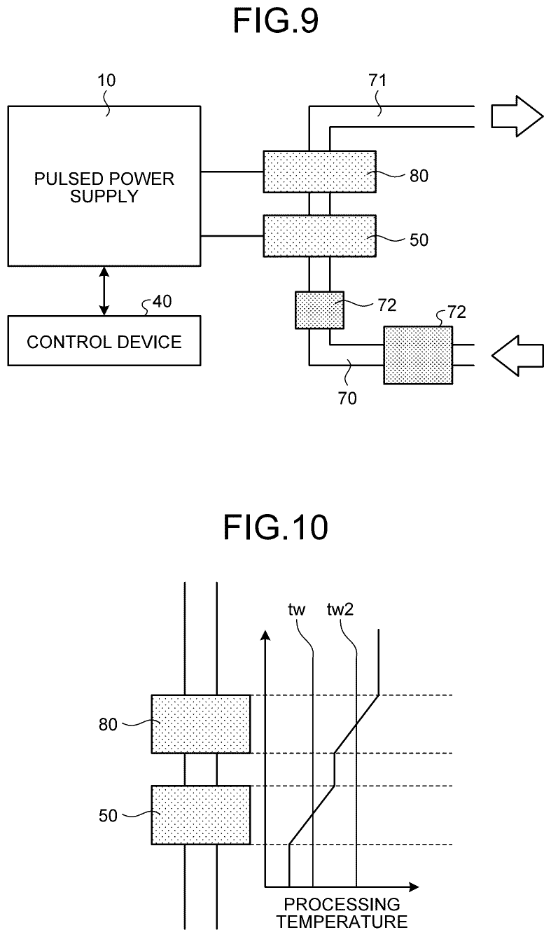

FIG. 9 is a schematic diagram illustrating an exemplary configuration of a pulsed electric field processing apparatus according to a second embodiment. In the second embodiment, a processing unit 80 as a second processing unit is added downstream of the processing unit 50 of the pulse electrolytic processing apparatus according to the first embodiment. Note that constituent elements that fulfill functions similar to the functions of those in the first embodiment are denoted by the same reference numerals as those in the first embodiment, and redundant description will be omitted.

The processing unit 80 has a configuration similar to that of the processing unit 50. That is, the processing unit 80 includes an electrode (not illustrated) that is a second electrode having a configuration similar to that of the electrode 51, an insulating member (not illustrated) having a configuration similar to that of the insulating member 54, and a processing chamber (not illustrated) that is a second processing chamber having a configuration similar to that of the processing chamber 55. A second pulse voltage output from the pulsed power supply 10 is applied to a high-voltage side electrode of the second electrode, and a second pulse current flows through the second processing chamber in accordance with the second pulse voltage and a resistance value of the processing target object inside the second processing chamber. The temperature computing unit 42 computes a second processing temperature tw2 that is a temperature of the processing target object in the second processing chamber on the basis of the resistance value and a preset calibration value. The resistance value is acquired from the second pulse voltage and the second pulse current flowing through the processing target object in the second processing chamber when the second pulse voltage is applied. The power supply control unit 41 adjusts the output from the pulsed power supply 10 on the basis of at least two of the processing temperature tw of the processing target object in the processing unit 50, the second processing temperature tw2, and a target temperature set downstream of the processing unit 80.

FIG. 10 is a schematic diagram illustrating an example of a temperature distribution of the pulsed electric field processing apparatus according to the second embodiment. In FIG. 10, a horizontal axis represents the processing temperature, and a vertical axis represents the position of the processing target object in the flow direction. The processing temperature of the processing target object increases while the processing target object passes through the processing unit 50, and the processing temperature of the processing target object further increases while the processing target object passes through the processing unit 80.

The power supply control unit 41 adjusts the output from the pulsed power supply 10 according to a difference between the processing temperature tw and the second processing temperature tw2. At this time, by doubling the difference between the processing temperature tw and the second processing temperature tw2, the temperature rise of the processing target object while the processing target object passes through the processing unit 50 and the processing unit 80 can be computed. As a result, the output from the pulsed power supply 10 can be adjusted so as to achieve a desired temperature rise. Alternatively, the target temperature for the processing target object downstream of the processing unit 80 may be set to adjust the output from the pulsed power supply 10 by comparing the target temperature with a value obtained by adding half of the power consumed by the processing unit 80 to the second processing temperature tw2. Therefore, by using at least two of the processing temperature tw, the second processing temperature tw2, and the target temperature set for the processing target object downstream of the processing unit 80, the output from the pulsed power supply 10 can be controlled with respect to either the upper limit value of the temperature or the value of the temperature rise of the processing target object. The pulsed power supply 10 outputs different voltage waveforms to the processing unit 50 and the processing unit 80 based on independent control. With such control, the second processing temperature tw2 can be brought closer to the target temperature with higher accuracy, Alternatively, the pulsed power supply 10 may output the same voltage waveforms to both the processing unit 50 and the processing unit 80. In this case, there is an advantage that the pulsed power supply 10 is simplified.

As described above, according to the second embodiment, the processing unit 80 is added downstream of the processing unit 50, and the temperature of the processing target object is controlled by using at least two of the processing temperature tw, the second processing temperature tw2, and the target temperature set for the processing target object downstream of the processing unit 80. Therefore, accuracy of the temperature control on the processing target object is further improved, and the processing quality of the processing target object can be further improved.

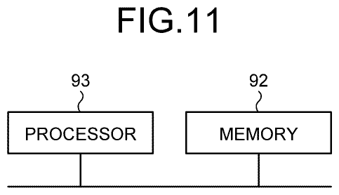

FIG. 11 is a block diagram illustrating an example of a hardware configuration that implements the control system of the first and second embodiments. The constituent elements of the control device 40, the state observation unit 90, and the machine learning unit 91 illustrated in FIG. 6 are implemented by a processor 93 and a memory 92.

The above configurations illustrated in the embodiments are examples of the contents of the present disclosure, and can be combined with other known techniques, and the above configurations can be partly omitted or changed without departing from the gist of the present disclosure.

### Reference Signs List

10 pulsed power supply; 11, 12 capacitor; 13, 14 switch; 15 direct-current power supply; 16 current limiter; 22 pulse voltage measurement device; 23 pulse current measurement device; 24 temperature measurement device; 40 control device; 41 power supply control unit; 42 temperature computing unit; 43 first anomaly detection unit; 44 second anomaly detection unit; 50, 80 processing unit; 51 electrode; 52 high-voltage electrode; 53 low-voltage electrode; 54 insulating member; 55 processing chamber; 70 upstream pipe; 71 downstream pipe; 72 heating unit; 90 state observation unit; 91 machine learning unit; 92 memory; 93 processor.

1. A pulsed electric field processing apparatus, comprising:
a pulsed power supply to generate a pulse voltage;
a pair of first electrodes to generate a pulse electric field by being applied with the pulse voltage;
a first processing chamber disposed between the pair of the first electrodes, to include a space through which a processing target object in a liquid state passes and in which the pulse electric field is generated;
a processor
to compute a first processing temperature that is a temperature of the processing target object in the first processing chamber on a basis of a resistance value and a preset calibration value, the resistance value being acquired from the pulse voltage and a pulse current flowing through the processing target object in the first processing chamber when the pulse voltage is applied to the first electrodes; and
to control the pulsed power supply on a basis of the first processing temperature and a preset target temperature.

2. The pulsed electric field processing apparatus according to claim 1,
wherein the processor further outputs a first anomaly signal on a basis of a comparison between the first processing temperature computed by the temperature computing unit and a preset set value or an average value of the first processing temperature in the past.

3. The pulsed electric field processing apparatus according to claim 1, wherein
the processor
performs control to change at least one of a pulse frequency and a pulse width of the pulse voltage without changing a voltage value of the pulse voltage.

4. The pulsed electric field processing apparatus according to claim 1, wherein
the pulsed power supply
includes a circuit in which a capacitor and a switch are connected in series, where a charging voltage of the capacitor is discharged to apply a pulse voltage to the pair of the first electrodes, and
the processor computes the pulse current on a basis of a charging current of the capacitor.

5. The pulsed electric field processing apparatus according to claim 4,
wherein the processor observes the charging voltage and the charging current of the capacitor as a state variable; and
learns a normal value of the charging voltage or a normal value of the charging current using a training data set created on a basis of the state variable and a physical property value of the processing target object.

6. The pulsed electric field processing apparatus according to claim 1, comprising
a temperature measurement device to measure a temperature of the processing target object, the temperature measurement device being disposed at least either upstream or downstream of the first processing chamber, and wherein
the processor outputs a second anomaly signal on a basis of a comparison between a temperature measured by the temperature measurement device and a temperature of the processing target object at least either upstream or downstream of the first processing chamber computed.

7. The pulsed electric field processing apparatus according to claim 1, comprising
a heater to heat the processing target object, the heater being provided upstream of the first processing chamber.

8. The pulsed electric field processing apparatus according to claim 1, comprising:
second electrodes to generate a pulse electric field by being applied with the pulse voltage from the pulsed power supply, the second electrodes being disposed downstream of the first electrodes; and
a second processing chamber disposed between a pair of the second electrodes, to include a space through which the processing target object passes and in which the pulse electric field is generated, wherein
the processor
computes a second processing temperature that is a temperature of the processing target object in the second processing chamber on a basis of a resistance value and a preset calibration value, the resistance value being acquired from the pulse voltage and a pulse current flowing through the processing target object in the second processing chamber when the pulse voltage is applied to the second electrodes, and
the processor controls the pulsed power supply on a basis of at least two of the first processing temperature, the second processing temperature, and a target temperature set for the processing target object downstream of the second processing chamber.
